(19) United States
(12) Patent Application Publication
(10) Pub. No.: US 2025/0099552 A1
(43) Pub. Date: Mar. 27, 2025

(54) PHARMACEUTICAL OR FODD SUPPLEMENT FORMULATION CONTAINING ALPHA-LACTALBUMIN AND BUTYRIC ACID OR A SALT THEREOF

(71) Applicant: KOLFARMA S.R.L., Genova (IT)

(72) Inventor: Paolo Mainardi, Genova (IT)

(73) Assignee: KOLFARMA S.R.L., Genova (IT)

(21) Appl. No.: 18/290,888

(22) PCT Filed: Jul. 20, 2022
(86) PCT No.: PCT/EP2022/070337

§ 371 Date: Jan. 22, 2024

(30) Foreign Application Priority Data
Jul. 23, 2021 (IT) 102021000019613

(51) Int. Cl. A61K 38/38; A61K 9/00; A61K 9/16; A61K 31/19

(52) U.S. Cl. CPC A61K 38/38; A61K 9/0053; A61K 9/1652; A61K 31/19

(57) ABSTRACT

A pharmaceutical or food supplement formulation in powder form for oral administration, comprising alpha-lactalbumin, butyric acid or a salt thereof with an alkaline or alkaline-earth metal and maltodextrins, in which the weight ratio of butyric acid or salt thereof: alpha-lactalbumin is between 1:4 and 1:1.5 and preferably the weight ratio of butyric acid or salt thereof: maltodextrins is between 1:3 and 1:2.

### FIELD OF APPLICATION

The present invention relates to the technical field of the pharmaceutical industry or food supplement industry.

In particular, the invention relates to a pharmaceutical or food supplement formulation containing alpha-lactalbumin and butyric acid or a salt thereof with an alkaline or alkaline-earth metal.

### PRIOR ART

Alpha-lactalbumin is known to be used as food supplement mainly because of its brain serotonergic action.

Italian Patent Application N. GE2006A000013 on behalf of the Applicant relates indeed to the use of alpha-lactalbumin in the treatment of neurological and neuropsychiatric pathologies, such as Parkinson's disease, depressive pathologies and epilepsy and European Patent N. EP 2 218 462 B1 relates to a pharmaceutical preparation comprising a SSRI or a SSNRI and alpha-lactalbumin for the treatment of depressive disorders.

Patent Application WO 2019/175274 of the present Applicant described the synergistic action of alpha-lactalbumin with short-chain fatty acids (SCFAs) in the treatment of disorders of the central nervous system, especially of those linked to serotonin deficiency, such as for example epilepsy, depression, and anxiety.

Among the SCFAs mentioned in said application, butyric acid and alkaline or alkaline-earth salts thereof are particularly preferred, and a formulation of granules of sodium butyrate in sachets is specifically exemplified, said formulation being intended to be administered in conjunction with a separate formulation of granules of alpha-lactalbumin in sachets.

The granules of sodium butyrate contain 300 mg sodium butyrate and 1500 mg hydrogenated palm oil, in addition to calcium carbonate (200 mg), maltodextrins (90 mg) and flavoring agent (10 mg). The granules were produced by nebulizing a mixture of sodium butyrate, hydrogenated palm oil and calcium carbonate through a nozzle of a cooling chamber at about −10° C. The granules thereby obtained have a sodium butyrate-based inner core and a coating based on hydrogenated palm oil and calcium carbonate.

The coating of hydrogenated palm oil and calcium carbonate is used for masking the very unpleasant taste and smell of sodium butyrate.

In fact, the unpleasant and bitter taste of butyrate makes it problematic incorporating it in a formulation in powder form, because, when it comes into contact with the bitter taste receptors, mainly located on the tip of the tongue, it stimulates a vomiting reaction.

On the other hand, a powder formulation containing butyrate to be dissolved or dispersed in water or other liquids for oral administration would be extremely useful for administering butyrate to children and adults with swallowing problems, who have difficulties in taking coated tablets, which represent the solution adopted so far in the known art for masking the unpleasant taste and smell of butyrate.

Patent application WO/2012/013495, for example, describes pharmaceutical formulations containing an SCFA, for example butyric acid, or a salt or ester o amide thereof, a soluble or water-dispersable dietary fiber, including inulin, pectins, dextrin and maltodextrin, at least one flavoring agent and one or more excipients. The description only provides examples of formulations in the form of coated tablets of calcium butyrate, wherein the coating has the function of masking the taste and smell of butyrate and of possibly giving gastroresistance. No examples of formulations in powder form are provided.

Patent application WO 2017/160345 describes pharmaceutical preparations for oral administration of sodium phenylbutyrate, an active substance suitable for the treatment of neurodegenerative disorders such as Parkinson's disease and characterized, like butyric acid and salts thereof, by an unpleasant smell. For masking the unpleasant smell of sodium phenylbutyrate, beads are proposed that comprise a central cellulose core covered by a layer containing sodium phenylbutyrate, that is in turn covered by a smell-masking coating based on dimethylaminoethyl methacrylate, butyl methacrylate and methyl methacrylate.

Patent WO 2011/112695 describes a nutritional composition comprising whey protein powder comprising whey protein micelles and leucine, wherein the leucine content in the composition is between 20% and 40% by weight dry matter and the weight ratio of added leucine to whey protein micelles is from 1:2 to 1:3. In this composition, the bitter taste of leucine is masked.

Patent application CN 106036140 discloses a feedstuff for piglets in the form of granules, comprising nineteen ingredients, among which whey protein concentrate (30-50 parts), maltodextrin (46-60 parts), sodium butyrate (3-5 parts) and an acidulant (4-15 parts). The main ingredients of this feedstuff are, however, flour (130-200 parts) and broken rice (230-350 parts). Such feedstuff is not in powder form and it is clearly not intended for administration to a human subject, but to an animal such as a piglet that does not display any vomiting reaction in response to a bitter and unpleasant taste like that of butyrate.

Patent application WO 2019/228851 discloses a dietary supplement providing for a source of butyrate with improved organoleptic properties, such a source of butyrate being a triglyceride, in which one or two hydroxy group(s) of glycerol is/are esterified with butyric acid and the remaining one(s) is/are esterified with a long chain fatty acid having 16 to 20 carbon atoms. This dietary supplement can also comprise prebiotics, such as inulin and FOS, sweeteners and emulsifiers.

Patent application WO 2012/013495 discloses a dietary composition containing a SCFA or an ester or amide thereof, in combination with a soluble or water-dispersible dietary fibre, such as e.g. inulin or maltodextrin, and a flavoring agent.

The problem underlying the present invention was to provide a pharmaceutical or food supplement formulation in powder form for oral administration to a human subject that might allow the simultaneous administration of butyric acid or an alkaline or alkaline-earth salt thereof and alpha-lactalbumin, masking the unpleasant taste and smell of butyric acid or salts thereof, without resorting to a coated table, so that the pharmaceutical or food supplement formulation might be taken even by children and subjects having swallowing problems.

### SUMMARY OF THE INVENTION

Said problem was solved, according to the invention, by a pharmaceutical or food supplement formulation in powder form for oral administration to a human subject, comprising alpha-lactalbumin, butyric acid or a salt thereof with an alkaline or alkaline-earth metal and maltodextrins, in which the weight ratio of butyric acid or said salt thereof: alpha-lactalbumin ranges between 1:4 and 1:1.5, preferably between 1:2 and 1:1.5.

By “pharmaceutical or food supplement formulation in powder form” a formulation in the form of a powder with a particle size lower than 1000 μm, typically 10-800 μm, is meant.

Preferably, in the formulation according to the invention the weight ratio of butyric acid or said salt thereof: maltodextrins ranges between 1:3 and 1:2.

The formulation may further comprise inulin, preferably in a weight ratio of between 1:4 and 1:1, preferably between 1:2 and 1:1.3, with respect to butyric acid or said salt thereof.

The formulation may further comprise fructoligosaccharides (FOS), preferably in a weight ratio of between 1:4 and 1:1.5, preferably between 1:2.7 and 1:1.8, with respect to butyric acid or said salt thereof.

The formulation may further comprise a flavoring agent, and/or a sweetening agent, and/or an emulsifying agent.

Preferably, the flavoring agent is contained in a percentage of between 0.1% and 3%, more preferably between 0.5% and 2%, by weight of the total weight of the formulation.

Preferred flavoring agents are banana flavoring and chocolate flavoring.

Preferably, the sweetening agent is contained in a percentage of between 0.1% and 3%, more preferably between 0.5 and 2%, by weight of the total weight of the formulation. A particularly preferred sweetening agent is sucralose.

Preferably, the emulsifying agent is contained in a percentage of between 0.05% and 1%, more preferably between 0.1% and 0.5%, by weight of the total weight of the formulation. Preferably, the emulsifying agent is a non-ionic surfactant, conveniently a polysorbate, in particular polysorbate 80. The formulation according to the present invention generally comprises the ingredients that are shown, in percentages by weight of the total weight of the formulation, in the following Table 1.

TABLE 1

| | |
|---|---|
| Alpha-lactalbumin | 20-30 |
| Butyric acid or alkaline or alkaline-earth salt thereof | 10-18 |
| Maltodextrins | 40-50 |
| Inulin | 7-15 |
| Fructoligosaccharides | 5-10 |
| Flavoring agent | 0-3 |
| Sweetening agent | 0-3 |
| Emulsifying agent | 0-1 |

Preferably, the formulation according to the present invention contains the above-mentioned ingredients in the percentages by weight of the total weight of the formulation shown in the following Table 2.

TABLE 2

| | |
|---|---|
| Alpha-lactalbumin | 22-27 |
| Butyric acid or alkaline or alkaline-earth salt thereof | 12-16 |
| Maltodextrins | 42-48 |
| Inulin | 8-13 |
| Fructoligosaccharides | 6-9 |
| Flavoring agent | 0.5-2 |
| Sweetening agent | 0.5-2 |
| Emulsifying agent | 0.1-0.5 |

Preferably, the formulation according to the invention is in powder form and is intended to be dissolved in water or in an aqueous beverage, typically in a volume of about 150-200 ml.

The formulation according to present invention provides a masking of the rancid smell and taste of butyrate that is surprising, since no document of the prior art reports an effect of masking the rancid smell and taste of butyrate or of other rancid-smelling substance by means of addition of alpha-lactalbumin and maltodextrins. In fact, the above-mentioned document WO 2011/112695 reports an effect of masking a bitter and non-rancid taste by using not alpha-lactalbumin or simple whey proteins, but specific whey protein micelles obtained according to the method described in WO 2007/110411.

### DETAILED DESCRIPTION

The present invention will be further described with reference to some examples which are provided by way of non-limiting illustration.

A formulation in powder form in 4 g sachets was prepared starting from the following ingredients, as described below in Example 1 and in Example 2.

### Example 1

Alpha-lactalbumin (ALAC) 1000 mg
Fructoligosaccharides (FOS) 300 mg
Inulin 400 mg
Sodium butyrate 625 mg
Banana flavoring (flavoring agent) 37 mg
Maltodextrins 1563 mg
Sucralose (sweetening agent) 60 mg
Polysorbate 80 (emulsifying agent) 15 mg
Total 4000 mg

The above-listed ingredients in powder form were mixed until homogeneous, and sachets for oral administration were filled with the resultant mixture.

### Example 2

Alpha-lactalbumin (ALAC) 1200 mg
Fructoligosaccharides (FOS) 250 mg
Inulin 400 mg
Sodium butyrate 625 mg
Banana flavoring (flavoring agent) 37 mg
Maltodextrins 1413 mg
Stevia powder extract (sweetening agent) 60 mg
Polysorbate 80 (emulsifying agent) 15 mg
Total 4000 mg

The above-listed ingredients in powder form were mixed until homogeneous, and sachets for oral administration were filled with the resultant mixture.

### Example 3

Alpha-lactalbumin (ALAC) 1000 mg
Fructoligosaccharides (FOS) 250 mg
Inulin 320 mg
Sodium butyrate 625 mg
Banana flavoring (flavoring agent) 70 mg
Maltodextrins 1697 mg
Sucralose (sweetening agent) 30 mg
Polysorbate 80 (emulsifying agent) 8 mg
Total 4000 mg

The above-listed ingredients in powder form were mixed until homogeneous, and sachets for oral administration were filled with the resultant mixture.

### Example 4 (Comparative)

Alpha-lactalbumin (ALAC) 700 mg
Fructoligosaccharides (FOS) 400 mg
Inulin 400 mg
Sodium butyrate 625 mg
Banana flavoring (flavoring agent) 37 mg
Maltodextrins 1763 mg
Sucralose (sweetening agent) 60 mg
Polysorbate 80 (emulsifying agent) 15 mg
Total 4000 mg

### Example 5 (Test of Organoleptic Evaluation)

The solution obtained by dissolving the content of a sachet of powder according to Examples 1 and 4 in 150 ml water underwent evaluation by a panel of 12 trained tasters, who were asked to give a score from 1 to 10 (1=minimum intensity; 10=maximal intensity) to the organoleptic characteristics specified in the following Table 3. The scores shown in the table are the average of the scores given by each taster.

TABLE 3

| Characteristic | Score Example 1 | Score Example 4 |
|---|---|---|
| Rancid smell | 1.5 | 4.5 |
| Rancid taste | 1.2 | 4.1 |
| Acrid taste | 1.6 | 4.7 |
| Sweetish aftertaste | 1.8 | 4.5 |
| Bitter taste | 1.5 | 4.3 |
| Taste of fat | 1.8 | 4.7 |

In the case of the formulation according to example 1, the intensity of the negative organoleptic characteristics that are typical of butyrates was very low and all the tasters confirmed the overall palatability of the solution.

On the contrary, for the formulation according to the comparative example 4, wherein the amount of alpha-lactalbumin is lower than the amount of alpha-lactalbumin in the formulation according to the present invention, the tasters distinctly perceived the presence of negative organoleptic characteristics, in particular the rancid taste and smell that are typical of butyrates.

1. A pharmaceutical or food supplement formulation in powder form for oral administration to a human subject, comprising
alpha-lactalbumin, butyric acid or a salt thereof with an alkaline or alkaline-earth metal and maltodextrins, in which a weight ratio of butyric acid or said salt thereof: alpha-lactalbumin is between 1:4 and 1:1.5.

2. The pharmaceutical or food supplement formulation according to claim 1, wherein a weight ratio of butyric acid or said salt thereof: maltodextrin is between 1:3 and 1:2

3. The pharmaceutical or food supplement formulation according to claim 1, wherein said formulation further comprises inulin.

4. The pharmaceutical or food supplement formulation according to claim 3, wherein inulin is present in a weight ratio of between 1:4 and 1:1 with respect to butyric acid or said salt thereof.

5. The pharmaceutical or food supplement formulation according to claim 4, wherein said formulation further comprises fructoligosaccharides (FOS).

6. The pharmaceutical or food supplement formulation according to claim 5, wherein said fructoligosaccharides are present in a weight ratio of between 1:4 and 1:1.5 with respect to butyric acid or said salt thereof.

7. The pharmaceutical or food supplement formulation according to claim 1, wherein said formulation further comprises a flavoring agent, in a percentage of between 0.1% and 3% by weight of the total weight of the formulation.

8. The pharmaceutical or food supplement formulation according to claim 1, wherein said formulation further comprises a sweetening agent in a percentage of between 0.1% and 3% by weight of the total weight of the formulation.

9. The pharmaceutical or food supplement formulation according to claim 1, wherein said formulation further comprises an emulsifying agent in a percentage of between 0.05% and 1% by weight of the total weight of the formulation.

10. The pharmaceutical or food supplement formulation according to claim 9, wherein said emulsifying agent is a non-ionic.

11. The pharmaceutical or food supplement formulation according to claim 1, wherein said weight ratio of butyric acid or said salt thereof: alpha-lactalbumin is between 1:2 and 1:1.5.

12. The pharmaceutical or food supplement formulation according to claim 4, wherein inulin is present in a weight ratio of between 1:2 and 1:1.3 with respect to butyric acid or said salt thereof.

13. The pharmaceutical or food supplement formulation according to claim 6, wherein said fructoligosaccharides are present in a weight ratio of between 1:2.7 and 1:1.8 with respect to butyric acid or said salt thereof.

14. The pharmaceutical or food supplement formulation according to claim 1, wherein said formulation further comprises a banana flavoring or a chocolate flavoring in a percentage of between 0.5% and 2% by weight of the total weight of the formulation.

15. The pharmaceutical or food supplement formulation according to claim 1, wherein said formulation further comprises a sweetening agent in a percentage of between 0.5 and 2%, by weight of the total weight of the formulation.

16. The pharmaceutical or food supplement formulation according to claim 8 wherein said sweetening agent is sucralose.

17. The pharmaceutical or food supplement formulation according to claim 1, wherein said formulation further comprises an emulsifying agent in a percentage of between between 0.1% and 0.5% by weight of the total weight of the formulation.

18. The pharmaceutical or food supplement formulation according to claim 10, wherein said a non-ionic surfactant is polysorbate 80.
